(19) United States
(12) Patent Application Publication
(10) Pub. No.: US 2005/0260275 A1
(43) Pub. Date: Nov. 24, 2005

(54) Encapsulated peptide copper complexes and compositions and methods related thereto

(75) Inventor: Leonard M. Patt, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: ProCyte Corporation, Redmond, WA (US)

(21) Appl. No.: 11/095,040

(22) Filed: Mar. 30, 2005

Related U.S. Application Data
(60) Provisional application No. 60/558,644, filed on Apr. 1, 2004.

(51) Int. Cl. A61K 38/16; A61K 9/16; A61K 9/50

(52) U.S. Cl. 424/490; 514/6

(57) ABSTRACT

This invention relates to compositions comprising encapsulated peptide copper complexes, and, additionally, to such compositions formulated for use as pharmaceutical and cosmetic products, as well as to medical devices that comprise such compositions.

### CROSS-REFERENCE TO RELATED APPLICATION

This application claims the benefit under 35 U.S.C. § 119(e) of U.S. Provisional Patent Application No. 60/558,644 filed Apr. 1, 2004, which provisional application is incorporated herein by reference in its entirety.

### BACKGROUND OF THE INVENTION

1. Field of the Invention

The present invention relates to compositions comprising encapsulated peptide copper complexes, and, additionally, to such compositions formulated for use as pharmaceutical and cosmetic products, as well as to medical devices that comprise such compositions.

2. Description of the Related Art

Copper is known to have many beneficial biological applications, including, as a few examples, stimulating the accumulation of collagen and elastin in wounds and damaged tissue (see, e.g., Maquart et al., J. Clin. Invest. 92:2368-2376 (1993); Maquart et al., FEBS Lett. 238(2):343-346 (1988); and Wegrowski et al., Life Sci. 51(13):1049-1056 (1992)) and in intact skin after topical application (see, e.g., Abdulghani et al., Disease Management and Clinical Outcomes 1(4):136-141 (1998)), modulating the activity of matrix metalloproteases (see, e.g., Simeon et al., J. Invest. Dermatol. 112(6):957-964 (1999)), increasing angiogenesis (see, e.g., Ahmed et al., Biomaterials 20:201-209 (1999); Hu, G. F., J. Cell. Biochem. 69:326-35 (1998); Lane et al., J. Cell. Biol. 125(4):929-943 (1994); and Raju et al., JNCI 69(5):1183-1188 (1982)), and increasing the rate and extent of wound healing (see, e.g., Counts et al, Federation of American Societies for Experimental Biology Journal 6[5], A1636 (1992); Downey et al., Surgical Forum 36:573-575(1985); Fish et al., Wounds 3:171-177 (1991); Mulder et al., Wound Repair and Regeneration 1:89 (1993); Swaim et al., Am. J. Vet Res. 57:394-399 (1996); and Swaim et al., J. Am. Anim. Hosp. Assoc. 29:519-525 (1993)).

Water-soluble ionic copper salts are generally ineffective, or even inhibitory, for such applications, usually due to irritation caused by application of the salt. The copper ion must be delivered in a biologically acceptable form. As an example, when copper is complexed with a biologically acceptable carrier molecule, such as a peptide or protein, it may then be effectively delivered to cells and tissues.

Specifically, peptide copper complexes, and compositions comprising the same, may be effective in this regard. Peptide copper complexes that are useful for wound healing and skin health are disclosed in U.S. Pat. Nos. 4,760,051; 4,665,054; 4,877,770; 5,135,913 and 5,348,943. Peptide copper complexes, beneficial for stimulating hair growth and preventing hair loss, are disclosed in U.S. Pat. Nos. 5,177,061; 5,214,032; 5,120,831; 5,550,183 and 5,538,945. Another beneficial application of peptide copper complexes is the prevention and healing of gastric ulcers, as disclosed in U.S. Pat. Nos. 5,145,838; 4,767,753 and 5,023,237. Yet another utility of such complexes is the healing of bone, as disclosed in U.S. Pat. No. 5,059,588.

Representative examples of methods for encapsulating pharmaceutical or cosmetic actives are disclosed in, but not limited to, the following issued United States Patents.

U.S. Pat. No. 6,572,892 to Loulalen et al. entitled “Cosmetic or dermopharmaceutical composition in the form of beads and methods for preparing same” describes an anhydrous solid composition comprising at least a hydrophobic wax, an oil and talcum, having the form of beads with sizes ranging from 1 to 10,000 microns.

U.S. Pat. No. 6,572,870 to Ribier et. al. entitled “Anhydrous cosmetic makeup composition containing a fatty phase” describes an anhydrous cosmetic makeup composition containing, in addition to a fatty phase formed from oils, fatty bodies and surfactants, and optionally waxes, a vesicular lipidic phase that contains at least one ionic or nonionic amphiphilic lipid and optionally additives.

U.S. Pat. No. 6,569,463 to Patel et al. entitled “Solid carriers for improved delivery of hydrophobic active ingredients in pharmaceutical compositions” describes solid pharmaceutical compositions for improved delivery of a wide variety of pharmaceutical active ingredients contained therein or separately administered. The disclosed encapsulation coat can include different combinations of pharmaceutical active ingredients, hydrophilic surfactant, lipophilic surfactants and triglycerides. In another disclosed embodiment, the solid pharmaceutical composition includes a solid carrier, the solid carrier being formed of different combinations of pharmaceutical active ingredients, hydrophilic surfactants, lipophilic surfactants and triglycerides.

U.S. Pat. No. 6,565,886 to Simonnet et al. entitled “Nanocapsules based on poly(alkylene adipate), process for their preparation and cosmetic or dermatological compositions containing them” describes nanocapsules consisting of a lipid center forming or containing a lipophilic active compound, and of a water-insoluble continuous envelope comprising at least one polyester of poly(alkylene adipate) type.

U.S. Pat. No. 6,565,873 to Shefer et al. entitled “Biodegradable bioadhesive controlled release system of nano-particles for oral care products” describes a controlled release system. The disclosed controlled release system is a nano-particle, having an average particle diameter of from about 0.01 microns to about 10 microns, which comprises a biodegradable solid hydrophobic core and a bioadhesive or mucoadhesive positively charged surface.

U.S. Pat. No. 6,548,690 to Mimoun entitled “Porous polymethylsilsesquioxane with adsorbent properties” describes a porous polymethylsilsesquioxane useful as an encapsulation matrix.

U.S. Pat. No. 6,548,569 to Williams et al. entitled “Medical devices and applications of polyhydroxyalkanoate polymers” describes biocompatible polyhydroxyalkanoates having controlled degradation rates, which are useful as slow release polymers.

U.S. Pat. No. 6,537,568 to Olejnik et al. entitled “Implant device with a retinoid for improved biocompatibility” describes an implant device, which incorporates a retinoid for improving the biocompatibility of the device in tissue.

U.S. Pat. No. 6,534,549 to Newton et al. entitled “Controlled release formulations” describes a method for producing a controlled release composition in which a film-forming composition comprising a mixture of a substantially water-insoluble film-forming polymer and amylose in a solvent system comprising (1) water and (2) a water-miscible organic solvent which on its own is capable of dissolving the film-forming polymer is contacted with an active material and the resulting composition dried.

U.S. Pat. No. 6,531,160 to Biatry et al. entitled “Microcapsules with an aqueous core containing at least one water-soluble cosmetic or dermatological active principle and cosmetic or dermatological compositions containing them” describes microcapsules with an aqueous core containing at least one water-soluble cosmetic or dermatological active principle, and with a polymeric and/or waxy envelope, in which the envelope consists of at least one polymer chosen from polycaprolactone, poly(3-hydroxybutyrate), poly(ethylene adipate), poly(butylene adipate), cellulose esters of at least one C₁-C₄ carboxylic acid, copolymers of styrene and of maleic anhydride, copolymers of styrene and of acrylic acid, styrene-ethylene/butylene-styrene block terpolymers, styrene-ethylene/propylene-styrene block terpolymers and terpolymers of ethylene, of vinyl acetate and of maleic anhydride, and/or of at least one wax chosen from beeswax, polyglycerolated beeswax, hydrogenated plant oils, paraffin with a melting point above 45° C., and silicone waxes.

U.S. Pat. No. 6,497,902 to Ma entitled “Ionically crosslinked hydrogels with adjustable gelation time” describes biocompatible hydrogels comprising at least one water-soluble polymer/copolymer; and at least one slow and/or fast dissolving and/or releasing divalent and/or multivalent cation-containing compound.

While a number of compositions comprising peptide copper complexes have been identified and described as having biologically beneficial utility, there remains a need in the art for peptide copper complex compositions that can more effectively, economically and easily be used for preparing pharmaceuticals, cosmetic products and medical devices. In particular, needed in the art, in this regard, are peptide copper complex compositions that are compatible with a wide range of formulation components, many of which, for example glycolic acids and the like, normally would be incompatible with peptide copper complexes. It has been discovered that encapsulation of peptide copper complexes allows their formulation and combination with other factors with which they normally, in the un-encapsulated form, would not be compatible. The present invention fulfills these needs and provides further related advantages.

### BRIEF SUMMARY OF THE INVENTION

In brief, the present invention is directed to compositions comprising encapsulated peptide copper complexes, having utility as pharmaceutical and cosmetic products, as well as medical devices.

Encapsulation as used herein means any composition or method of entrapping or surrounding an effective amount of a peptide copper complex as described herein in such a manner that the peptide copper complex is released slowly, altered in its interactions with other formulation components, altered in its solubility, or otherwise altered in its basic physical properties.

As previously noted, peptide copper complexes, and compositions comprising the same, have beneficial utility for, as some examples, skin health and appearance; wound healing; hair, bone and tissue growth; and hair loss prevention. Accordingly, the present invention, in another embodiment, is directed to a disclosed composition that further comprises an inert and physiologically acceptable carrier or diluent, thus being suitable for use as a pharmaceutical or cosmetic product. In a related embodiment, disclosed is a medical device that comprises a composition of the present invention.

These and other aspects of the present invention will be evident upon reference to the following detailed description of the invention.

### DETAILED DESCRIPTION OF THE INVENTION

Definitions

As defined herein, the term “encapsulating matrix” refers to a compound or mixture of compounds that encapsulate or enclose a peptide copper complex in a composition, thereby protecting the associated peptide copper complex from chemical or physical interactions with other formulation components.

As defined herein, an “encapsulated peptide copper complex” is a peptide copper complex enclosed by an encapsulating matrix in such a way that the peptide copper complex is protected from, for example, the effects of solvents, preservatives, or other components present in a formulation is such a way that the stability of the copper peptide is enhanced.

As defined herein, the abbreviations for the naturally occurring amino acids are:

| | | |
|---|---|---|
| Alanine | Ala | A |
| Arginine | Arg | R |
| Asparagine | Asn | N |
| Aspartic Acid | Asx | D |
| Cysteine | Cys | B |
| Glycine | Gly | G |
| Glutamine | Gln | Q |
| Glutamic Acid | Glu | E |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As noted above, in one embodiment of the present invention, disclosed is an encapsulated peptide copper complex. In more specific embodiments, the peptide copper complex is glycyl-L-histidyl-L-lysine:copper(II) (“GHK-Cu”), L-alanyl-L-histidyl-L-lysine:copper(II) (“AHK-Cu”), L-valyl-L-histidyl-L-lysine:copper(II) (“VHK-Cu”), L-leucyl-L-histidyl-L-lysine:copper(II) (“LHK-Cu”), L-isoleucyl-L-histidyl-L-lysine:copper(II) (“IHK-Cu”), L-phenylalanyl-L-histidyl-L-lysine:copper(II) (“FHK-Cu”), L-prolyl-L-histidyl-L-lysine:copper(II) (“PHK-Cu”), L-seryl-L-histidyl-L-lysine:copper(II) (“SHK-Cu”), or L-threonyl-L-histidyl-L-lysine:copper(II) (“THK-Cu”).

As used herein, the expression “peptide copper complex” generally refers to a coordination compound comprising a peptide molecule and a copper(II) ion non-covalently complexed with the peptide. As is well understood in the art, copper (II) designates a copper ion having a valence of 2 (i.e., Cu⁺²). The peptide molecule is a chain of two or more amino acid units or amino acid derivative units covalently bonded together. Generally, an amino acid consists of an amino group, a carboxyl group, a hydrogen atom, and an amino acid side-chain moiety—all bonded, in the case of an alpha-amino acid, to a single carbon atom that is referred to as an alpha-carbon. The amino acid units of the present invention may be provided by amino acids other than alpha-amino acids. For example, the amino acids may be beta- or gamma-amino acids, such as the following: 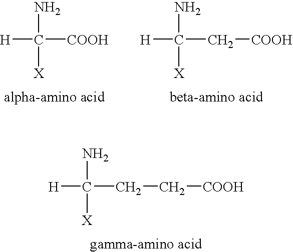
alpha-amino acid beta-amino acid gamma-amino acid where X is the amino acid side-chain moiety bonded, along with the amino group and hydrogen, to an alpha-, beta-, or gamma-carbon atom.

As another example, the amino acids of the peptide include, but are not limited to, naturally occurring alpha-amino acids. The naturally occurring amino acids shown are all in the L configuration, referring to the optical orientation of the alpha carbon or other carbon atom bearing the amino acid side chain. A peptide molecule of the present invention may also comprise amino acids that are in the D optical configuration, or a mixture thereof.

Representative amino acid derivatives include those set forth in Table 1 below.

TABLE 1
Amino Acid Derivatives

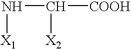

Where X₂ = H or the following moieties:
—(CH₂)ₙCH₃ where n = 1-20
—(CH₂)ₙCH(CH₃)(CH₂)ₘCH₃ where n, m = 0-20 (when n = 0,
m ≠ 0 or 1 and when n = 1, m ≠ 0)
—(CH₂)ₙNH₂ where n = 1-20 (n ≠ 4)
—(CH₂)ₙCONH₂ where n = 3-20
—(CH₂)ₙCOOH where n = 3-20

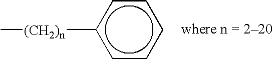

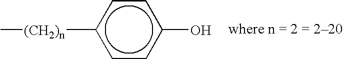

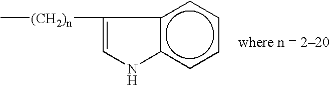

—(CH₂)ₙSH where n = 2-20
—(CH₂)ₙS(CH₂)ₘCH₃ where n, m = 1-20 (when n = 2, m ≠ 0)
—(CH₂)ₙCH₂OH where n = 1-20
—(CH₂)ₙCH(CH₃)OH where n = 1-20
And where X₁ = H or the following moieties:
—(CH₂)ₙCH₃ where n = 0-20
—(CH₂)ₙCH(CH₃)(CH₂)ₘCH₃ where n, m = 0-20

Histidine derivatives of this invention include compounds having the structure: 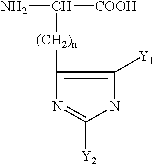
where n=1-20, and Y₁ and Y₂ are independently selected from alkyl moieties containing from 1-12 carbon atoms or an aryl moiety containing from 6-12 carbon atoms. In certain embodiments, n is 1, Y₂ is methyl, and Y₁ is H (i.e., 3-methyl histidyl) or Y₂ is H and Y₁ is methyl (i.e., 5-methyl histidine).

As used herein, “alkyl” means a straight chain or branched, cyclic or noncyclic, substituted or unsubstituted, saturated or unsaturated aliphatic hydrocarbon containing from 1 to 18 carbon atoms. Representative saturated straight chain alkyls include methyl, ethyl, n-propyl and the like; while saturated branched alkyls include isopropyl, sec-butyl, isobutyl, tert-butyl, isopentyl, and the like. Representative, saturated cyclic alkyls include cyclopropyl, cyclobutyl, cyclopentyl, —CH₂cyclohexyl, and the like; while unsaturated cyclic alkyls include cyclopentenyl, cyclohexenyl, and the like. Unsaturated alkyls contain at least one double or triple bond between adjacent carbon atoms (referred to as an “alkenyl” or “alkynyl,” respectively). Representative alkenyls include ethylenyl, 1-butenyl, isobutylenyl, 2-methyl-2-butenyl, and the like; while representative alkynyls include acetylenyl, 2-butynyl, 3-methyl-1-butynyl, and the like.

Also, as used herein, “aryl” means an aromatic carbocyclic moiety such as phenyl or naphthyl, and may be substituted or unsubstituted. “Arylalkyl,” as used herein, means an alkyl having at least one alkyl hydrogen atom replaced with a substituted or unsubstituted aryl moiety, such as benzyl (i.e., —CH₂phenyl, —(CH₂)₂phenyl, —(CH₂)₃phenyl, —CH(phenyl)₂, and the like).

In certain embodiments, n is 1, Y₂ is methyl and Y₁ is H (i.e., 3-methyl histidyl) or Y₂ is H and Y₁ is methyl (i.e., 5-methyl histidine).

Similarly, arginine derivatives of this invention include compounds having the structure: 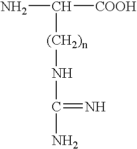
where n=1-20 (excluding n=3)

A peptide copper complex of the present invention may have the formula [R₁—R₂—R₃]:copper(II) where R₃ is at least one amino acid or amino acid derivative, as defined above, bonded to R₂ by a peptide bond. Where R₃ is a single amino acid or amino acid derivative, then the peptide of the peptide copper complex is generally classified as a tripeptide. As another example of a peptide copper complex of the present invention having the formula [R₁—R₂—R₃]:copper(II), R₃ is a chemical moiety bonded to the R₂ moiety by an amide bond. The expression “chemical moiety,” as used herein and with reference to R₃, includes any chemical moiety having an amino group capable of forming an amide bond with the carboxyl terminus of R₂ (i.e., the carboxyl terminus of histidine, arginine, or derivatives thereof).

As a more particular example, where R₃ is a chemical moiety bonded to the R₂ moiety by an amide bond, R₃ is —NH₂, an alkylamino moiety having from 1-20 carbon atoms, or an arylamino moiety having from 6-20 carbon atoms. As used herein, an “alkylamino moiety” encompasses alkyl moieties containing an amino moiety, wherein the alkyl moiety is as defined above, and includes, but is not limited to, octyl amine and propyl amine. Similarly, an “arylamino moiety” encompasses aryl moieties containing an amino moiety, wherein the aryl moiety is as defined above, and includes, but is not limited to, benzylamine and benzyl-(CH₂)₁₋₁₄-amine. Further examples of suitable chemical moieties having amino groups capable of forming an amide linkage with the carboxyl terminus of R₂ include polyamines such as spermine and sperimidine.

It should be understood that R₃ may include more than one chemical moiety. For example, additional amino acids or amino acid derivatives may be bonded to the above-described peptide copper complexes comprising tripeptides to yield peptide copper complexes comprising peptides having four or more amino acids and/or amino acid derivatives. For purposes of illustration, Table 2, shown below, presents various representative examples of peptide copper complexes of the present invention.

TABLE 2
Representative Peptide-Copper Complexes
Examples of [R₁-R₂]:copper(II)

| | |
|---|---|
| glycyl-histidine:copper | alanyl-histidine:copper |
| glycyl-(3-methyl)histidine:copper | alanyl-(3-methyl)histidine:copper |
| glycyl-(5-methyl)histidine:copper | alanyl-(5-methyl)histidine:copper |
| glycyl-arginine:copper | alanyl-arginine:copper |
| (N-methyl)glycine-histidine:copper | (N-methyl)glycine-arginine:copper |

Examples of [R₁-R₂-R₃]:copper(II)
where R₃ is Chemical Moiety Linked by Amide Bond

| | |
|---|---|
| glycyl-histidyl-NH₂:copper | glycyl-arginyl-NH₂:copper |
| glycyl-(3-methyl)histidyl- | alanyl-(3-methyl)histidyl- |
| NH₂:copper | NH₂:copper |
| glycyl-arginyl-NH₂:copper | alanyl-arginyl-NH₂:copper |
| (N-methyl)glycine-histidyl- | (N-methyl)glycine-arginyl- |
| NH₂:copper | NH₂:copper |
| glycyl-histidyl-NHoctyl:copper | glycyl-arginyl-NHoctyl:copper |

Examples of [R₁-R₂-R₃]:copper(II)
where R₃ is Amino Acid or Amino Acid
Derivative Linked by Peptide Bond

| | |
|---|---|
| glycyl-histidyl-lysine:copper | glycyl-arginyl-lysine:copper |
| glycyl-(3-methyl)histidyl- | glycyl-(5-methyl)histidyl- |
| lysine:copper | lysine:copper |
| alanyl-histidyl-lysine:copper | alanyl-arginyl-lysine:copper |
| alanyl-(3-methyl)histidyl- | alanyl-(5-methyl)histidyl- |
| lysine:copper | lysine:copper |
| glycyl-histidyl- | glycyl-arginyl- |
| phenylalanine:copper | phenylalanine:copper |
| glycyl-(3-methyl)histidyl- | glycyl-(5-methyl)histidyl- |
| phenylalanine:copper | phenylalanine:copper |
| alanyl-histidyl- | alanyl-arginyl- |
| phenylalanine:copper | phenylalanine:copper |
| alanyl-(3-methyl)histidyl- | alanyl-(5-methyl)histidyl- |
| phenylalanine:copper | phenylalanine:copper |
| glycyl-histidyl-lysyl- | glycyl-arginyl-lysyl- |
| phenylalanyl- | phenylalanyl- |
| phenylalanyl:copper | phenylalanyl:copper |
| glycyl-(3-methyl)histidyl- | glycyl-(5-methyl)histidyl- |
| lysyl-phenylalanyl- | lysyl-phenylalanyl- |
| phenylalanyl:copper | phenylalanyl:copper |
| (N-methyl)glycyl-histidyl- | (N-methyl)glycyl-arginyl- |
| lysine:copper | lysine:copper |
| valyl-histidyl-lysine:copper | glycyl-histidyl-lysyl-prolyl- |
| prolyl-histidyl-lysine:copper | phenylalanyl-proline:copper |
| glycyl-D-histidyl- | Leucyl-histidyl-lysine:copper |
| L-lysine:copper | |
| seryl-histidyl-lysine:copper | |

Further examples of peptide copper complexes encompassed by the present invention are disclosed in U.S. Pat. Nos. 4,665,054; 4,760,051; 4,767,753; 4,810,693; 4,877,770; 5,023,237; 5,059,588; 5,118,665; 5,120,831; 5,164,367; 5,177,061; 5,214,032; 5,538,945; 5,550,183; and 6,017,888, all of which are incorporated herein by reference in their entirety.

Examples of the peptide copper complex derivatives, encompassed by the present invention, include, but are not limited to, those disclosed and described in the above-cited U.S. Patents that are directed to peptide copper complexes, as well as those disclosed and described in the published PCT application having the International Publication Number WO 94/03482, which is incorporated herein by reference in its entirety.

The synthesis of the above-disclosed peptide copper complexes is described in detail in the above-referenced patents. For example, the peptides of the peptide copper complexes disclosed herein may be synthesized by either solution or solid phase techniques known to one skilled in the art of peptide synthesis. The general procedure involves the stepwise addition of protected amino acids to build up the desired peptide sequence. The resulting peptide may then be complexed to copper (at the desired molar ratio of peptide to copper) by dissolving the peptide in water, followed by the addition of copper chloride or other suitable copper salt and adjusting the pH to greater than 4.0. The peptide copper complex thus formed may be used as a solution or as a dry powder after, for example, freeze-drying or spray drying.

The molar ratio of peptide to copper in the peptide copper complex thereof ranges from 1:1 to 3:1 and has a pH of about 4.0 to about 8.0. In yet further, more specific embodiments, the peptide copper complex is present at a concentration ranging from about 0.05% to about 25%; from about 0.05% to about 2%; and from about 0.1% to about 0.5%, respectively.

In yet another embodiment of the present invention, the peptide moiety of the peptide copper complex may also be of natural origin. In this embodiment, the peptide is formed by the hydrolysis of naturally occurring proteins, polypeptides, or larger peptides of either plant, microbial, or animal origin. Hydrolysis may be by enzymatic treatment or by acid or base hydrolysis. The copper complex of this type of peptide copper complex is formed by addition of a suitable copper salt to the aqueous solution of the peptide. Alternatively, the peptide copper complex may be formed during the manufacturing of a formulation by separate additions of the peptide and copper salt in a suitable solvent.

As described above, the encapsulated peptide copper complex composition of the present invention comprises an encapsulating matrix, in addition to the peptide copper complex.

Representative examples of encapsulation technology are described in the above cited patents, such as U.S. Pat. Nos. 6,572,892; 6,572,870; 6,569,463; 6,565,886; 6,566,873; 6,548,690; 6,548,569 and 6,537,568. One skilled in the art would readily recognize suitable encapsulation technology. In this invention, at least one of such encapsulating matrices is added to a peptide copper complex to form the encapsulated peptide copper complex.

In the case of topical application, representative encapsulating matrices include those that would also supply additional skin conditioning and treatment compounds to the skin. These would be comprised of oils, fats, triglycerides, emulsifying agents, and the like. Also representative are encapsulation ingredients comprised of bio-erodable polymers. Bio-erodable polymers are polymers which breakdown over time after application to the body either by chemical hydrolysis or enzymatic action.

Encapsulation is accomplished by means well known to one skilled in the art and varies with the nature of the encapsulation matrix. Encapsulation can be accomplished by specialized mixing techniques such as high speed homogenization or sonication of the components. Additional methods of forming encapsulated peptide copper complexes are disclosed in the above mentioned issued U.S. Patents, which are incorporated herein by reference in their entireties.

In an additional embodiment of this invention, the encapsulated peptide copper complex may also contain a suitable and effective preservative or mixture of preservatives. Any of the commonly used preservatives in cosmetic or medical formulations may be used to preserve the encapsulated peptide copper complexes. Representative preservatives are benzyl alcohol, benzoic acid, chlorophesin, phenoxyethanol, any of the parabens, and the like. Also representative are the “formaldehyde donor” preservatives such as diazolidinyl urea and imidazolidinyl urea.

The present invention, in another embodiment, is directed to a disclosed preserved and chemically stable composition that is formulated as an emulsion and topically applied to skin. In this embodiment, a disclosed composition further comprises an emulsifying agent, a surfactant, a thickening agent, an excipient, or a mixture thereof. Accordingly, the above-disclosed composition may be in the form of a liquid, lotion, cream, gel, emulsion, or microemulsion.

Also, one skilled in the art will appreciate that the above-disclosed encapsulating matrix compositions may comprise ingredients other than those listed above, such as, for example, an active drug substance.

In another embodiment of the present invention, the compositions of the present invention, adapted for topical application to the skin, may also contain at least one active cosmetic ingredient, in addition to the peptide copper complex. Active cosmetic ingredients, as defined herein, are compounds that provide benefits to the skin and/or desirable properties to cosmetic formulations. Some examples of active ingredients are sunscreens and tanning agents, skin conditioning agents, skin protectants, emollients and humectants. Other representative active ingredients are known to those of ordinary skill in the art as cosmetic actives, such as retinol, retinoids, various phytochemicals, and the like. Such other active ingredients may or may not be encapsulated.

The present invention, in another embodiment, is directed to a disclosed composition consisting of an encapsulated peptide copper complex suspended in an oil or wax or combination thereof. These would be comprised of oils, fats, triglycerides, emulsifying agents, and the like. Also representative are encapsulation ingredients comprised of bio-erodable polymers.

The present invention, in a related aspect, is also directed to medical devices that comprise a disclosed preserved and chemically stable composition. One non-limiting example of such a device is a sterile gauze pad, impregnated with a disclosed composition in the form of a gel or solution for application to a wound.

Encapsulated peptide copper complexes show utility in cosmetic formulations, medical preparations, and medical devices. For example, in a cosmetic formulation, the encapsulated peptide copper complex can be combined with other cosmetic actives as described above to lessen the signs of aging skin such as fine lines and wrinkles or hyperpigmentation. In pharmaceutical preparations or medical devices, the encapsulated peptide copper complexes can show utility by their ability to stimulate collagen and other components of the extracellular matrix important to tissue repair and rebuilding.

Any of the utility previously shown for the peptide copper complexes, and cited previously or known to one skilled in the art, would be expected to be shown by encapsulated peptide copper complexes and would be enhanced by being formulated with components which normally could not be present in the absence of the encapsulation. For example, encapsulated peptide copper complex could be formulated with alpha-glycolic acids or beta-glycolic acids to provide exfoliation of skin or encapsulated copper peptide could be formulated in a moisturizing oil such as squalane to provide moisturization in combination with the utility of the peptide copper complex.

The following examples, which illustrate the preparation, characterization, and utility of certain embodiments of the present invention, are provided for the purpose of illustration, not limitation.

### EXAMPLES

### Example 1

An encapsulated peptide copper complex is manufactured by compounding glycyl-histidyl-lysine copper complex with Polyethylene glycol polymers, non-ionic surfactant, a phospholipids, and sorbitol. This material is emulsified by standard techniques into cyclopentasiloxane to produce a deep blue colored clear solution. The emulsion is stable and does not settle out.

This solution has utility as an anhydrous (without water) formulation containing a peptide copper complex in an encapsulating matrix. Peptide copper complex which was not in an encapsulating matrix would not be soluble in an anhydrous formulation.

### Example 2

The encapsulated peptide copper complex of Example 1 is combined with other formulation ingredients in a cream base to form a blue cream useful for the moisturization of skin.

### Example 3

The encapsulated peptide copper complex of Example 1 is combined with pure squalane to form a blue suspension useful for the moisturization of skin.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

1. An encapsulated peptide copper complex comprising a peptide copper complex and an encapsulating matrix.

2. The encapsulated peptide copper complex of claim 1 wherein the peptide copper complex is glycyl-L-histidyl-L-lysine:copper(II).

3. The encapsulated peptide copper complex of claim 1 wherein the peptide copper complex is L-alanyl-L-histidyl-L-lysine:copper(II).

4. The encapsulated peptide copper complex of claim 1 wherein the peptide copper complex is L-valyl-L-histidyl-L-lysine:copper(II).

5. The encapsulated peptide copper complex of claim 1 wherein the peptide copper complex is L-leucyl-L-histidyl-L-lysine:copper(II).

6. The encapsulated peptide copper complex of claim 1 wherein the peptide copper complex is L-isoleucyl-L-histidyl-L-lysine:copper(II).

7. The encapsulated peptide copper complex of claim 1 wherein the peptide copper complex is L-phenylalanyl-L-histidyl-L-lysine:copper(II).

8. The encapsulated peptide copper complex of claim 1 wherein the peptide copper complex is L-prolyl-L-histidyl-L-lysine:copper(II).

9. The encapsulated peptide copper complex of claim 1 wherein the peptide copper complex is L-seryl-L-histidyl-L-lysine:copper(II).

10. The encapsulated peptide copper complex of claim 1 wherein the peptide copper complex is L-threonyl-L-histidyl-L-lysine:copper(II).

11. The encapsulated peptide copper complex of claim 1 wherein the peptide portion of the peptide copper complex comprises an amino acid, a series of amino acids, or an amino acid derivative.

12. The encapsulated peptide copper complex of claim 1 wherein the peptide portion of the peptide copper complex comprises a peptide formed by the hydrolysis of naturally occurring proteins, polypeptides, or larger peptides of either plant, microbial, or animal origin.

13. A composition comprising the encapsulated peptide copper complex of claim 1 wherein the composition is in the form of a liquid, lotion, cream, gel, emulsion, or microemulsion.

14. A composition comprising the encapsulated peptide copper complex of claim 1 wherein the composition is in the form of an oil or wax or mixture thereof.

15. The composition of claim 13 further comprising an active drug substance.

16. The composition of claim 13 further comprising an active cosmetic substance.

17. A medical device comprising the encapsulated peptide copper complex of claim 1.

18. A method for treating aging skin utilizing the encapsulated peptide copper complex of claim 1.

19. A method for treating wounds utilizing the encapsulated peptide copper complex of claim 1.

20. A method for treating hyperpigmentation utilizing the encapsulated peptide copper complex of claim 1.

21. A method for cosmetic treatment of skin, comprising contacting the skin in need thereof with an effective amount of the encapsulated peptide copper complex of claim 1.

22. A method for treating aged and photodamaged skin, comprising contacting the skin in need thereof with an effective amount of the encapsulated peptide copper complex of claim 1.

23. A method for treating wrinkles skin, comprising contacting the skin in need thereof with an effective amount of the encapsulated peptide copper complex of claim 1.

24. A method for treating hyperpigmentation, comprising contacting the skin in need thereof with an effective amount of the encapsulated peptide copper complex of claim 1.
